(19) United States
(12) Patent Application Publication
(10) Pub. No.: US 2016/0074462 A1
(43) Pub. Date: Mar. 17, 2016

(54) Method of Treating HCV

(71) Applicant: AbbVie Inc., North Chicago, IL (US)

(72) Inventors: Sandeep Dutta, Lincolnshire, IL (US); Rajeev Menon, Buffalo Grove, IL (US); Akshanth Polepally, Waukegan, IL (US); Tianli Wang, Lake Bluff, IL (US)

(73) Assignee: ABBVIE INC., North Chicago, IL (US)

(21) Appl. No.: 14/849,836

(22) Filed: Sep. 10, 2015

Related U.S. Application Data
(60) Provisional application No. 62/048,917, filed on Sep. 11, 2014.

(51) Int. Cl. A61K 38/07; A23L 1/29; A61K 31/427; A61K 31/513; A61K 38/05

(52) U.S. Cl. CPC A61K 38/07; A61K 31/513; A61K 38/05; A61K 31/427; A23L 1/293; A23V 2002/00

(57) ABSTRACT

This application features methods of treating HCV using Compound 1/r, Compound 2 and/or Compound 3.

### FIELD OF THE TECHNOLOGY

This application relates to methods of treating HCV using Compound 1/r, Compound 2 and/or Compound 3.

### BACKGROUND

Patients with chronic hepatitis C virus (HCV) infection are at risk for developing progressive liver fibrosis, cirrhosis, portal hypertension, hepatocellular carcinoma and decompensated liver disease. HCV can be cured with antiviral therapy, reducing the risk of morbidity and mortality associated with end-stage liver disease.

For approximately a decade, HCV genotype 1-infected patients have been treated with peginterferon/ribavirin dual therapy resulting in sustained virologic response rates (SVR) of approximately 40-50%. However, substantial limitations to efficacy and tolerability remain as many users suffer from side effects, and viral elimination from the body is often incomplete.

### DETAILED DESCRIPTION

Compound 1, Compound 2 and Compound 3 are potent direct acting agents (DAAs) against HCV.

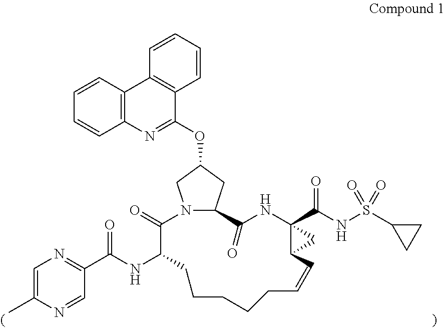 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

is known as (2R,6S,13aS,14aR,16aS,Z)-N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclop entadecine-14a-carboxamide. The synthesis and formulation of Compound 1 are described in U.S. Patent Application Publication Nos. 2010/0144608 and 2011/0312973, respectively. Compound 1 is typically used with ritonavir. As used herein, “Compound 1/ritonavir” and “Compound 1/r” refer to the combination of Compound 1 and ritonavir, or co-administration of Compound 1 and ritonavir.

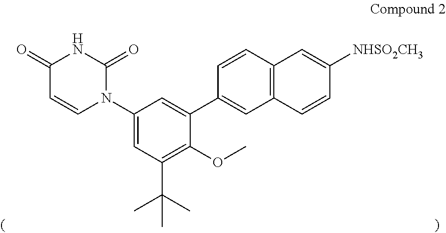

is known as N-(6-(3-tert-butyl-5-(2,4-dioxo-3,4-dihydropyrimidin-1(2H)-yl)-2-methoxyphenyl)naphthalen-2-yl)methanesulfonamide and is described in International Application Publication No. WO2009/039127.

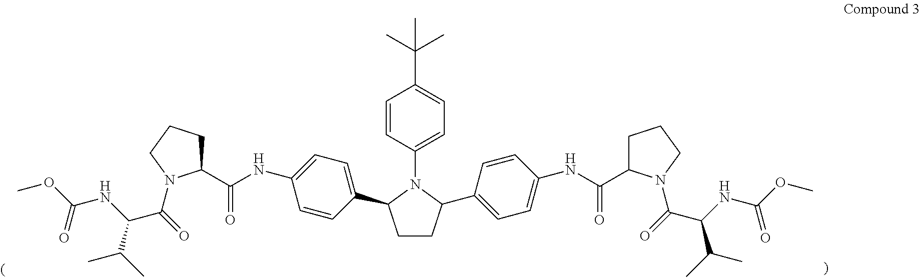

is known as dimethyl (2S ,2′S)-1,1′-((2S,2′S)-2,2′-(4,4′-((2S ,5S)-1-(4-tert-butylphenyl)pyrrolidine-2,5,diyl)bis(4,1-phenylene))bis(azanediyl)bis(oxomethylene)bis(pyrrolidine-2,1-diyl)bis(3-methyl-1-oxobutane-2,1-diyl)dicarbamate, and is described in U.S. Publication No. 2010/0317568.

The combination of Compound 1/r, Compound 2 and Compound 3, the combination of Compound 1/r and Compound 3, as well as the combination of Compound 1/r and Compound 2, have been shown to be effective against HCV genotype 1. High SVR rates can be achieved when patients infected with HCV genotype 1 are treated with these DAA combos in an interferon-free, short-duration regimen (e.g., in a regimen consisting of 8- or 12-week).

Food effect on the drug exposure (e.g., AUC) of Compound 1/r, Compound 2 and Compound 3 was not known. Food effect on the drug exposure of these compounds was also not considered predictable.

It was discovered that food increased the exposure (AUC) of Compound 3, Compound 1, ritonavir, and Compound 2 by up to 127%, 367%, 63%, and 53%, respectively, relative to the fasting state. The increase in exposure was similar regardless of meal type (e.g., high-fat vs. moderate-fat) or calorie content (500-600 kcal vs. 1000 kcal). Therefore, the combination of Compound 1/r, Compound 2 and Compound 3, the combination of Compound 1/r and Compound 3, as well as the combination of Compound 1/r and Compound 2, can be taken with food without regard to the specific fat or calorie content.

Accordingly, according to one aspect of the invention, the present invention features methods of treating HCV, comprising administering Compound 1/r, Compound 2 and Compound 3, to a patient in need thereof, wherein these compounds are taken with food.

In another aspect, the present invention features methods of treating HCV, comprising administering Compound 1/r, Compound 2 and Compound 3, to a patient in need thereof, wherein these compounds are taken with food but without regard to fat and calorie content of the food.

In yet another aspect, the present invention features methods of treating HCV, comprising administering to a patient in need thereof two Compound 1/r/Compound 3 tablets once daily and one Compound 2 tablet twice daily, wherein all of the tablets are taken with food, and wherein each Compound 1/r/Compound 3 tablet comprises 75 mg Compound 1, 50 mg ritonavir and 12.5 mg Compound 3, and each Compound 2 tablet comprises 250 mg Compound 2.

In yet another aspect, the present invention features methods of treating HCV, comprising administering to a patient in need thereof two Compound 1/r/Compound 3 tablets once daily and one Compound 2 tablet twice daily, wherein all of the tablets are taken with food but without regard to fat and calorie content of the food, and wherein each Compound 1/r/Compound 3 tablet comprises 75 mg Compound 1, 50 mg ritonavir and 12.5 mg Compound 3, and each Compound 2 tablet comprises 250 mg Compound 2.

In yet another aspect, the present invention features methods of treating HCV, comprising administering Compound 1/r and Compound 3 to a patient in need thereof, wherein these compounds are taken with food.

In another aspect, the present invention features methods of treating HCV, comprising administering Compound 1/r and Compound 3 to a patient in need thereof, wherein these compounds are taken with food but without regard to fat and calorie content of the food.

In yet another aspect, the present invention features methods of treating HCV, comprising administering to a patient in need thereof two Compound 1/r/Compound 3 tablets once daily, wherein the tablets are taken with food, and wherein each Compound 1/r/Compound 3 tablet comprises 75 mg Compound 1, 50 mg ritonavir and 12.5 mg Compound 3.

In yet another aspect, the present invention features methods of treating HCV, comprising administering to a patient in need thereof two Compound 1/r/Compound 3 tablets once daily, wherein the tablets are taken with food but without regard to fat and calorie content of the food, and wherein each Compound 1/r/Compound 3 tablet comprises 75 mg Compound 1, 50 mg ritonavir and 12.5 mg Compound 3.

In yet another aspect, the present invention features methods of treating HCV, comprising administering Compound 1/r and Compound 2 to a patient in need thereof, wherein these compounds are taken with food.

In another aspect, the present invention features methods of treating HCV, comprising administering Compound 1/r and Compound 2 to a patient in need thereof, wherein these compounds are taken with food but without regard to fat and calorie content of the food.

In any aspect of the invention, the patient can be, for example, infected with HCV genotype 1.

In any aspect of the invention, the patient can be, for example, infected with HCV genotype 1a.

In any aspect of the invention, the patient can be, for example, infected with HCV genotype 1b.

In any aspect of the invention, the patient can be, for example, a treatment-naïve patient infected with HCV genotype 1.

In any aspect of the invention, the patient can be, for example, a treatment-naïve patient infected with HCV genotype 1a.

In any aspect of the invention, the patient can be, for example, a treatment-naïve patient infected with HCV genotype 1b.

In any aspect of the invention, the patient can be, for example, an interferon null responder infected with HCV genotype 1.

In any aspect of the invention, the patient can be, for example, an interferon null responder infected with HCV genotype 1a.

In any aspect of the invention, the patient can be, for example, an interferon null responder infected with HCV genotype 1b.

In any aspect of the invention, the patient can be, for example, an interferon partial responder infected with HCV genotype 1.

In any aspect of the invention, the patient can be, for example, an interferon partial responder infected with HCV genotype 1 a.

In any aspect of the invention, the patient can be, for example, an interferon partial responder infected with HCV genotype 1b.

In any aspect of the invention, the patient can be, for example, an interferon relapser infected with HCV genotype 1.

In any aspect of the invention, the patient can be, for example, an interferon relapser infected with HCV genotype 1a.

In any aspect of the invention, the patient can be, for example, an interferon relapser infected with HCV genotype 1b.

In any aspect and preference of the invention, the treatment can, for example, be interferon-free (i.e., does not include administration of interferon) and last for 8 weeks.

In any aspect and preference of the invention, the treatment can, for example, be interferon-free and last for 9 weeks.

In any aspect and preference of the invention, the treatment can, for example, be interferon-free and last for 10 weeks.

In any aspect and preference of the invention, the treatment can, for example, be interferon-free and last for 11 weeks.

In any aspect and preference of the invention, the treatment can, for example, be interferon-free and last for 12 weeks.

In any aspect and preference of the invention, the treatment can, for example, be interferon-free and ribavirin-free, and last for 8 weeks.

In any aspect and preference of the invention, the treatment can, for example, be interferon-free and ribavirin-free, and last for 9 weeks.

In any aspect and preference of the invention, the treatment can, for example, be interferon-free and ribavirin-free, and last for 10 weeks.

In any aspect and preference of the invention, the treatment can, for example, be interferon-free and ribavirin-free, and last for 11 weeks.

In any aspect and preference of the invention, the treatment can, for example, be interferon-free and ribavirin-free, and last for 12 weeks.

In any aspect and preference of the invention, the treatment can, for example, be interferon-free and ribavirin-free, and last for 24 weeks.

In any aspect and preference of the invention, the treatment can, for example, be interferon-free but comprise administration of ribavirin, and last for 8 weeks.

In any aspect and preference of the invention, the treatment can, for example, be interferon-free but comprise administration of ribavirin, and last for 9 weeks.

In any aspect and preference of the invention, the treatment can, for example, be interferon-free but comprise administration of ribavirin, and last for 10 weeks.

In any aspect and preference of the invention, the treatment can, for example, be interferon-free but comprise administration of ribavirin, and last for 11 weeks.

In any aspect and preference of the invention, the treatment can, for example, be interferon-free but comprise administration of ribavirin, and last for 12 weeks.

In any aspect and preference of the invention, the treatment can, for example, be interferon-free but comprise administration of ribavirin, and last for 24 weeks.

The patient treated according to any aspect, example or preference of the invention can, for example, be infected with genotype 2, 3, 4, 5, or 6, instead of genotype 1. The patient treated according to any aspect, example or preference of the invention can, for example, have cirrhosis, or be non-cirrhotic. The patient treated according to any aspect, example or preference of the invention can, for example, be co-infected with HIV and said another drug is an anti-HIV agent. The patient treated according to any aspect, example or preference of the invention can, for example, be a liver transplant recipient.

As used herein, Compound 1, Compound 2 and Compound 3 encompass their respective pharmaceutically acceptable salts.

It should be understood that the above description and the following examples are given by way of illustration, not limitation. Various changes and modifications within the scope of the present application will become apparent to those skilled in the art from the present description.

What is claimed is:

1. A method of treating HCV, comprising administering Compound 1/r, Compound 2 and Compound 3, to a patient in need thereof, wherein all of the compounds are administered with food.

2. A method of treating HCV, comprising administering Compound 1/r, Compound 2 and Compound 3, to a patient in need thereof, wherein all of the compounds are taken with food without regard to fat and calorie content of the food.
